(19) United States
(12) Patent Application Publication
(10) Pub. No.: US 2009/0060983 A1
(43) Pub. Date: Mar. 5, 2009

(54) Method And Composition For Making An Orally Disintegrating Dosage Form

(76) Inventors: Frank J. Bunick, Randolph, NJ (US); Joe Luber, Quakertown, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/847,444

(22) Filed: Aug. 30, 2007

(51) Int. Cl. A61K 9/14; A61K 31/00; A61K 9/20; A61K 9/68; A61P 43/00

(52) U.S. Cl. 424/440; 424/464; 424/484; 514/789

(57) ABSTRACT

The present invention relates to a process for making orally disintegrating dosage forms and means for packaging such dosage forms.

### SUMMARY OF THE INVENTION

The present invention relates to a process for making orally disintegrating dosage forms and means for packaging such dosage forms.

### BACKGROUND

Pharmaceuticals intended for oral administration are typically provided in solid form as tablets, capsules, pills, lozenges, or granules. Tablets are swallowed whole, chewed in the mouth, or dissolved in the oral cavity. Soft tablets that either are chewed or dissolve in the mouth are often employed in the administration of pharmaceuticals where it is impractical to provide a tablet for swallowing whole. With chewable tablets, the act of chewing helps to break up the tablet particles as the tablet disintegrates and may increase the rate of absorption by the digestive tract. Soft tablets are also advantageous where it is desirable to make an active ingredient available topically in the mouth or throat for both local effects or systemic absorption. Soft tablets are also utilized to improve drug administration in pediatric and geriatric patients. Soft tablets designed to disintegrate in the mouth prior to swallowing are particularly useful for improving compliance of pediatric patients.

Generally, soft tablets are made by compaction of a mixture of tabulating compounds including an active ingredient, flavoring, binders, etc. The mixture is fed into a die cavity of a tablet press and a tablet is formed by applying pressure. Hardness of the resulting tablet is a direct function of the compaction pressure employed and the compatibility of the ingredients in the formulation. A softer tablet, having an easier bite-through, may be prepared by employing reduced compaction pressures. The resulting tablet is softer, but also more fragile, brittle, and easily chipped.

Soft tablets designed to disintegrate in the mouth without chewing are disclosed by Cousin et al., in U.S. Pat. No. 5,464,632, and Wehling et al., in U.S. Pat. Nos. 5,223,264 and 5,178,878. While these soft tablets for oral administration advantageously disintegrate completely in the mouth prior to swallowing, they have the disadvantage of being highly friable, requiring costly specialized handling and packaging in order to prevent breakage.

Orally dissolving, orally disintegrating or quick dissolving oral dosage forms prepared using the addition and removal of solvents or through a lyophilization process in order to create a highly porous dosage form structure such as disclosed by Yamanouchi Pharma Co in U.S. Pat. No. 6,589,554 and Janseen Pharmaceutica in U.S. Pat. No. 6,224,905 disadvantageously involve complex and costly processing steps.

Several workers in the field have described chewable tablets comprising an active ingredient and a fatty or polymeric binder material. PCT Application No. WO 93/13758, for example, describes tablets made by combining and compressing a meltable binder, excipients, and a pharmaceutically active ingredient into a tablet, melting the binder in the tablet, and the solidifying the binder. During the melting step, the binder, a material such as a natural fat or polyethylene glycol, flows and fills in minor cracks within the tablet. If a coating is desired on the tablet, it must be coated with a coating material in a separate step.

U.S. Pat. No. 4,684,534 discloses a chewable tablet having a harder outer shell and a softer interior. The tablet is made from agglomerates comprising a carbohydrate and a small amount of a carbohydrate binder such as maltodextrin, in addition to the active ingredient. The agglomerates are compressed into a tablet, resulting in the harder outer shell surrounding the softer interior. The hardness of the outer shell is on the order of 6 to 18 kp.

U.S. Pat. No. 5,662,849 is directed to a method and apparatus for making a compressed dosage form. The compressed dosage form is compacted into a tablet directly into a product tray.

U.S. Pat. No. 6,258,381 is directed to a tablet and process in which a granular agglomerate is heated to melt a binder component only at or near the surface, and then cooled, such that the melted binder solidifies into a tablet having a substantially continuous phase on the outside of the tablet.

U.S. Pat. No. 6,932,979 is directed to a soluble, rubber-containing, coated chewable tablet. The tablet is prepared by mixing pulverant chewable components with molten fat or wax components. These components along with a syrup component produce a crumbly material that is cooled and milled to desired particle size, and then compressed into a tablet. The dosage form is not formed directly in a package or component thereof.

It has now been discovered that an orally disintegrating tablet can be made from a mixture comprising at least one active ingredient and a binder having a melting point of about 20 to about 160° C. A granular agglomerate is formed from the mixture, dispensed into a unit dosage package and heated to melt the binder partially or substantially throughout the granular agglomerate. The granular agglomerate is then cooled such that the melted binder solidifies into a fused aggregate portion. The resulting dosage form acquires the shape of the recess in which the agglomerate was deposited. A tablet made by such a method has the additional advantage of using taste-masked coated particulates or granules that require a high degree of plasticizer customarily introduced into such coatings to avoid rupture or cracking under compression since minimal or no compression force is used in the process herein.

### DETAILED DESCRIPTION OF THE INVENTION

It is an object of the present invention to provide a method and apparatus for forming an orally disintegrating dosage unit directly in the package for the dosage unit without the use or with the minimal use of solvents. The method provides for the formation of a tablet within a tablet package such as a blister package, within a manufacturing mold which is recycled during processing, or within a preformed (i.e. compressed, molded, deposited, extruded or formed) edible form. The process of the present invention includes providing a tablet package having an open-ended cavity generally in the shape of the desired tablet. A pre-measured volume of tableting feedstock material is deposited within the cavity. The tableting feedstock material is heated within the open-ended cavity so as to form the desired tablet. The package may then be sealed for ultimate distribution and sale. In an alternate embodiment the package is sealed prior to the heating step. The tablet is made from a mixture comprising one or more active ingredients, one or more binders and at least one carbohydrate or carbohydrate alcohol.

Suitable active ingredients include pharmaceuticals, minerals, vitamins and other nutraceuticals. Suitable pharmaceuticals include analgesics, decongestants, expectorants, antitussives, antihistamines, gastrointestinal agents, diuretics, bronchodilators, motion sickness agents, migraine treatment agents, antiemetics, antiflatulants, appetite suppressants, antifungals, oral care agents, osteoporosis treatments, sleep-inducing agents and mixtures thereof. Preferred pharmaceuticals for use as the active ingredient include acetaminophen, ibuprofen, flurbiprofen, naproxen, diclofenac, aspirin, pseudoephedrine, phenylpropanolamine, phenylephrine, chlorpheniramine maleate, clofedianol, dextromethorphan, diphenhydramine, domperidone, famotidine, loperamide, ranitidine, cimetidine, astemizole, terfenadine, fexofenadine, cetirizine, antacids, mixtures thereof and pharmaceutically acceptable salts thereof. More preferably, the active ingredient is selected from the group consisting of acetaminophen, ibuprofen, pseudoephedrine, phenylephrine, dextromethorphan, diphenhydramine, chlorpheniramine, calcium carbonate, magnesium hydroxide, magnesium carbonate, magnesium oxide, aluminum hydroxide, simethicone, mixtures thereof, and pharmaceutically acceptable salts thereof.

Suitable oral care agents include breath fresheners, tooth whiteners, antimicrobial agents, tooth mineralizers, tooth decay inhibitors, topical anesthetics, mucoprotectants, and the like.

Suitable flavorants for use in the dosage form include menthol, peppermint, mint flavors, fruit flavors, chocolate, vanilla, bubblegum flavors, coffee flavors, liqueur flavors and combinations and the like.

Examples of suitable gastrointestinal agents include antacids such as calcium carbonate, magnesium hydroxide, magnesium oxide, magnesium carbonate, aluminum hydroxide, sodium bicarbonate, dihydroxyaluminum sodium carbonate; stimulant laxatives, such as bisacodyl, cascara sagrada, danthron, senna, phenolphthalein, aloe, castor oil, ricinoleic acid, and dehydrocholic acid, and mixtures thereof; H2 receptor antagonists, such as famotadine, ranitidine, cimetidine, nizatidine; proton pump inhibitors such as omeprazole or lansoprazole; gastrointestinal cytoprotectives, such as sucraflate and misoprostol; gastrointestinal prokinetics, such as prucalopride, antibiotics for H. pylori, such as clarithromycin, amoxicillin, tetracycline, and metronidazole; antidiarrheals, such as diphenoxylate and loperamide; glycopyrrolate; antiemetics, such as ondansetron, analgesics, such as mesalamine.

In one embodiment of the invention, the active ingredient may be selected from bisacodyl, famotadine, ranitidine, cimetidine, prucalopride, diphenoxylate, loperamide, lactase, mesalamine, bismuth, antacids, and pharmaceutically acceptable salts, esters, isomers, and mixtures thereof.

In another embodiment, the active ingredient may be selected from analgesics, anti-inflammatories, and antipyretics: e.g. non-steroidal anti-inflammatory drugs (NSAIDs), including propionic acid derivatives: e.g. ibuprofen, naproxen, ketoprofen and the like; acetic acid derivatives: e.g. indomethacin, diclofenac, sulindac, tolmetin, and the like; fenamic acid derivatives: e.g. mefanamic acid, meclofenamic acid, flufenamic acid, and the like; biphenylcarbodylic acid derivatives: e.g. diflunisal, flufenisal, and the like; and oxicams: e.g. piroxicam, sudoxicam, isoxicam, meloxicam, and the like. In one embodiment, the active ingredient is selected from propionic acid derivative NSAID: e.g. ibuprofen, naproxen, flurbiprofen, fenbufen, fenoprofen, indoprofen, ketoprofen, fluprofen, pirprofen, carprofen, oxaprozin, pranoprofen, suprofen, and pharmaceutically acceptable salts, derivatives, and combinations thereof. In another embodiment of the invention, the active ingredient may be selected from acetaminophen, acetyl saicylic acid, ibuprofen, naproxen, ketoprofen, flurbiprofen, diclofenac, cyclobenzaprine, meloxicam, rofecoxib, celecoxib, and pharmaceutically acceptable salts, esters, isomers, and mixtures thereof.

The active ingredient(s) are present in the mixture in a therapeutically effective amount, which is an amount that produces the desired therapeutic response upon oral administration and can be readily determined by one skilled in the art. In determining such amounts, the particular compound being administered, the bioavailability characteristics of the active ingredient, the dose regime, the age and weight of the patient, and other factors must be considered.

If the active ingredient has an objectionable taste, it may be coated with a taste masking coating, as known in the art. Examples of suitable taste masking coatings are described in U.S. Pat. No. 4,851,226, U.S. Pat. No. 5,075,114, and U.S. Pat. No. 5,489,436. Commercially available taste masked active ingredients may also be employed. For example, acetaminophen particles which are encapsulated with ethylcellulose or other polymers by a coacervation process may be used in the present invention. Coacervation-encapsuilated acetaminophen may be purchased commercially from Eurand America, Inc. Vandalia, Ohio, or from Circa Inc., Dayton, Ohio. Additional suitable methods for applying taste-masked coatings are well known in the art and include but are not limited to fluid bed coating, complex coacervation, spray drying, and spray congealing as disclosed in, for example, U.S. Pat. Nos. 4,851,226, 5,653,993, 5,013,557, and 6,569,463, respectively.

The active ingredient or ingredients may be present in the dosage form in any form. For example, the active ingredient may be dispersed at the molecular level, e.g. melted or dissolved, within the dosage form, or may be in the form of particles, which in turn may be coated or uncoated. If the active ingredient is in form of particles, the particles (whether coated or uncoated) typically have an average particle size of about 1 micron to about 2000 microns. In one embodiment, such particles are crystals having an average particle size of about 1 micron to about 300 microns. In yet another embodiment, the particles are granules or pellets having an average particle size of about 50 microns to about 2000 microns, e.g. from about 50 microns to about 1000 microns or from about 100 microns to about 800 microns.

In one embodiment the dosage form comprises one portion of active ingredient in an immediate release form and a second portion of the same or a different active ingredient in a modified release form.

In one embodiment the active ingredient is in the form of a micro-gel bead, which is liquid filled in semi-solid filled. The micro-gel beads are added as a portion of the powder matrix. The orally disintegrating form of this invention has the added advantage of not using a compression step, allowing for the use of liquid or semisolid filled particles or beads which are deformable, since they will not rupture upon compression. These beads may be coated with gelling substances such as but not limited to gelatin, gellan gum, xanthan gum, agar, locust bean gum, carageenan; polymers or polysaccharides such as but not limited to sodium alginate, calcium alginate, hypromellose, hydroxypropyl cellulose and pullulan; and starches; with or without the addition of plasticizers such as but not limited to glycerin, polyethylene glycol, propylene glycol, triacetin, triethyl citrate and tributyl citrate. The active ingredient may be dissolved, suspended or dispersed in a filler material such as but not limited to high fructose corn syrup, sugars, glycerin, polyethylene glycol, propylene glycol, or oils such as but not limited to vegetable oil, oilve oil, or mineral oil.

In one embodiment of this dosage form, the active ingredient is coated with a polymer coating for taste-masking or other purposes that does not require the use of a high level of plasticizers. Plasticizers can be used in particle coatings, for example in taste-masked coatings or sustained release coatings, in order to prevent rupture upon compression. One advantage of this dosage form is that there is no compression step which may compromise the integrity of the coating. In this embodiment the total percentage of plasticizer, by weight of the coating, is less than about 20 percent, e.g. less than about 10 percent, e.g. less than 5 percent. In one embodiment the coating is substantially free of plasticizers, defined as less than 5 percent, e.g. less than 1 percent of plasticizers by weight of the coating.

In certain embodiments in which modified release of the active ingredient is desired, the active ingredient may optionally be coated with a known release-modifying coating. This advantageously provides an additional tool for modifying the release profile of active ingredient from the dosage form. For example, the dosage form may contain coated particles of one or more active ingredients, in which the particle coating confers a release modifying function, as is well known in the art. Examples of suitable release modifying coatings for particles are described in U.S. Pat. Nos. 4,173,626; 4,863,742; 4,980,170; 4,984,240; 5,286,497; 5,912,013; 6,270,805; and 6,322,819. Commercially available modified release active ingredients may also be employed. For example, acetaminophen particles, which are encapsulated with release-modifying polymers by a coacervation process, may be used in the present invention. Such coacervation-encapsulated acetaminophen is commercially available from, for example, Eurand America, Inc. or Circa Inc.

The binder is a material capable of thermal deformation and have a melting point in the range of about 20 to about 160° C., preferably about 40 to about 140° C., more preferably about 55 to about 100° C. The binder can be crystalline or amorphous and has the capability to resolidify upon melting. Examples of suitable binders include fats such as cocoa butter, hydrogenated vegetable oil such as palm kernel oil, cottonseed oil, sunflower oil, and soybean oil, mono, di, and triglycerides, phospholipids, waxes such as Carnauba wax, spermaceti wax, beeswax, candelilla wax, shellac wax, microcrystalline wax, and paraffin wax, water soluble polymers such as polyethylene glycol, polycaparactone, suitable fatty acid esters including sucrose fatty acid esters, mono, di, and triglycerides, glyceryl behenate, glyceryl palmitostearate, glyceryl monostearate, glyceryl tristearate, glyceryl trilaurylate, glyceryl myristate, GlycoWax-932, lauroyl macrogol-32 glycerides, and stearoyl macrogol-32 glycerides, polyethylene oxides and derivatives, and sucrose esters. Preferably, the binder is selected from hydrogenated vegetable oil, polyethylene glycol, waxes, and mixtures thereof. In one embodiment more than one binder is used in the dosage form of this invention.

The active ingredient or ingredients are typically capable of dissolution upon contact with a fluid such as water, stomach acid, intestinal fluid or the like. In one embodiment, the dissolution characteristics of the active ingredient meet USP specifications for immediate release tablets containing the active ingredient. In embodiments in which it is desired for the active ingredient to be absorbed into the systemic circulation of an animal, the active ingredient or ingredients should be capable of dissolution upon contact with a fluid such as water, gastric fluid, intestinal fluid or the like. In one embodiment, the dissolution characteristics of the active ingredient meet USP specifications for immediate release tablets containing the active ingredient. For example, for acetaminophen chewable tablets, USP 24 specifies that in pH 5.8 phosphate buffer, using USP apparatus 2 (paddles) at 75 rpm, at least 75% of the acetaminophen contained in the dosage form is released therefrom within 45 minutes after dosing, and for ibuprofen tablets, USP 24 specifies that in pH 7.2 phosphate buffer, using USP apparatus 2 (paddles) at 50 rpm, at least 80% of the ibuprofen contained in the dosage form is released therefrom within 60 minutes after dosing. See USP 24, 2000 Version, 19-20 and 856 (1999). In another embodiment, the dissolution characteristics of the active ingredient may be modified: e.g. controlled, sustained, extended, retarded, prolonged, or delayed.

A particularly preferred binder is polyethylene glycol (PEG) having at least 95% by weight of the PEG particles less than 100 microns as measured by conventional means such as light or laser scattering or sieve analysis and a molecular weight between 3350 and 8000 Daltons.

The amount of binder present in the mixture is proportional to the particle size of the binder where in the event the binder has up to 95% by weight of the binder in the dosage form has particle size of less than about 100 microns as measured by conventional means such as light or laser scattering or sieve analysis, a range of 10-20% of binder is appropriate, alternatively, in the event the binder has more than 50% by weight of the binder having a particle size between about 100 and about 400 microns as measured by sieve analysis, a range of 15-40% of binder is preferred. The lower particle size contributes a higher surface area within the dosage form, wherein the binder contributes a greater binding effect when heated.

Another essential component is at least one carbohydrate or carbohydrate alcohol selected from the group consisting of dextrose, sucrose, erythritol, mannitol, sorbitol, maltitol, xylitol, lactose, isomalt, starch hydrolysates, which include dextrins, dextrates, and maltodextrins, and the like, and mixtures thereof. The carbohydrate contributes to the dissolvability and mouthfeel of the dosage form, and also by aiding in distributing the dry binder across a broader surface area, diluting and cushioning the active ingredient. The carbohydrate may be present at level of about 5 percent to about 95 percent of the dosage form, e.g. about 20 percent to about 90 percent or about 40 percent to about 80 percent of the dosage form.

The mixture may contain other conventional ingredients, such as fillers, which include conventional dry binders like cellulose, cellulosic derivatives, polyvinyl pyrrolidone, hydroxypropylcellulose starch, modified starch, and mixtures thereof, and in particular microcrystalline cellulose; sweeteners like aspartame, acesulfame potassium, sticralose and saccharin; and lubricants, such as magnesium stearate, stearic acid, talc, and waxes. The mixture may also incorporate pharmaceutically acceptable adjuvants, including, for example, preservatives; flavors; acidulants such as but not limited to citric acid, malic acid, fumaric acid; sensates such as cooling agents and warming agents; textural modifiers such as hypromellose, hydroxypropyl cellulose, alginates, pullulan, and pectin; salivation inducing agents; antioxidants; surfactants; and coloring agents.

In one embodiment the method of producing the orally disintegrating form is substantially free of the use of solvents. In this embodiment the flowable powder matrix material is substantially free of solvents, and the filling process of the matrix into the blister cavity, manufacturing mold or edible form is substantially free of solvents. In this embodiment substantially free is defined as less than about 5 percent, e.g. less than about 1 percent or less than about 0.5 percent by weight of the orally disintegrating tablet or portion. Solvents may include but are not limited to water, organic solvents such as but not limited to alcohols, chlorinated solvents, hexanes, or acetone; or gaseous solvents such as but not limited to nitrogen, carbon dioxide or supercritical fluids.

The mixture of active ingredient, binder, carbohydrate and any optional ingredients is formed into a fused agglomerate as an orally disintegrating tablet in a process described below. Preferably, the granular agglomerate is prepared such that the tablet is relatively soft, i.e., capable of dissolving in the mouth or being chewed. The hardness of the tablet is preferably up to about 3 kiloponds per square centimeter (kp/cm²). More preferably, the hardness of the tablet is up to about 2, most preferably less than 1 kp/cm². In one embodiment the density of the orally disintegrating tablet or tablet portion is less than about 0.9 g/cc, e.g. less than about 0.8 g/cc, e.g. less than about 0.7 g/cc.

Hardness is a term used in the art to describe the diametral breaking strength as measured by conventional pharmaceutical hardness testing equipment, such as a Schleuniger Hardness Tester. In order to compare values across different size tablets, the breaking strength must be normalized for the area of the break. This normalized value, expressed in kp/cm², is sometimes referred in the art as tablet tensile strength. A general discussion of tablet hardness testing is found in Leiberman et al., Pharmaceutical Dosage Forms—Tablets, Volume 2, 2^{nd} ed., Marcel Dekker Inc., 1990, pp. 213-217, 327-329.

A more preferred test for hardness of an orally disintegrating tablet of the present invention relies upon a Texture Analyser TA-XT2i that is fitted with a 7 millimeter diameter flat faced probe and setup to measure and report compression force in grams. The probe moves at 0.5 millimeters per second to a depth of penetration of 2 millimeters. The maximum compression force is recorded. The measured forces recorded for orally dissolvable tablets made in accordance with the present invention preferably ranges from approximately 700 grams to about 6000 grams with a deviation of ±_{—}500, up to at most 10,000 grams.

The components of the orally dissolvable tablets having the greatest impact on measured hardness are particles size and amount of the binder, the amount, type, and particle size of the carbohydrate (i.e. dextrose or sucrose) or carbohydrate alcohol (i.e. sorbitol or mannitol), and the type and characteristics of the active drug (i.e. APAP or Ibuprofen), including its state (i.e. crystal shape, coated particle, etc.), melting point, and particle size;, and whether the tablet was tamped or not, and the tablet shape.

As the particle size of the binder is decreased, less heat (in terms of time of heating and temperature) is needed to fuse the agglomerate to achieve the same hardness. In one embodiment the particle size of the of carbohydrate or carbohydrate alcohol can influence the level of binder used, wherein a higher particle size of carbohydrate or carbohydrate alcohol provides a lower surface area and subsequently requires a lower level of binder. In one embodiment, wherein the carbohydrate or carbohydrate alcohol is greater than 50% of the blend by weight of the blend and the mean particle size of the carbohydrate or carbohydrate alcohol is greater than 100 μm, then the binder is 10-20 percent by weight of the blend.

The melting point of the active ingredient can have an impact on the temperature used during the fusing or heating step and the type of binder used. In one embodiment, the melting point of the binder can be less than the melting point of the active. In another embodiment, the melting point of the active can be the same or lower than the melting point of the binder, in which case during the fusing or heating step, both may melt and create a eutectic or various bridges of active and binder between the other materials in the tablet form upon cooling.

In one embodiment, the fusing or heating temperature is above the melting point of the binder and below the melting point of the active ingredient. In one embodiment wherein ibuprofen is the active ingredient, the fusing temperature is between 30° C. and 60° C.

In one embodiment, the particle size of the active ingredient causes more void spaces to be present in the tablet blend, wherein a higher particle size of the active subsequently requires a lower level of binder. In one embodiment, wherein the active ingredient or coated active ingredient is greater than 50% of the blend by weight of the blend, and the mean particle size of the carbohydrate or carbohydrate alcohol is greater than 100 μm, then the binder is 10-20 percent by weight of the blend. In one embodiment, wherein the mean particle size of the total powder blend is between about 100 μm and about 300 μm, then binder is 10-20 percent by weight of the blend.

In one embodiment, the orally disintegrating form is tamped after filling the powder blend but prior to the heating or fusing step in order to remove air from the powder blend. In one embodiment the tamping step is not enough pressure or force to hold the tablet shape together. In one embodiment the method of producing the orally disintegrating tablet is substantially free of a tamping step. In one embodiment the tamping step is conducted using a force less than 0.3 kiloNewtons.

In one embodiment a vibratory step is utilized, wherein vibration is added after filling of the flowable powder blend but prior to the heating or fusing step, in order to remove air from the dosage form. In one embodiment a vibration with the frequency from about 1 Hz to about 50 KHz is added with amplitude from 1 micron to 5 mm peak-to-peak to allow for the flowable powder to settle into the blister cavity or dosage form cavity.

The shape of the tablet can have an impact on the measured hardness. For example, in one embodiment, a convex shaped orally disintegrating tablet face, produced by a concave shaped blister may have a higher level of hardness or a lower friability value than a flat faced orally disintegrating tablet.

In one embodiment the internal temperature of the dosage form at the center of the powder form is between 35° C. and 70° C. at the median time of the heating step (i.e. 2.5 minutes during a 5 minute heating step) when measured using a thermocouple temperature measuring sensor, such as a thermocouple Type K commercially available from the Hewitt Industries.

The crystal shape of the active ingredient can have an impact on the level of binder used. For example, in one embodiment, a more spherical shape type of crystal for the active ingredient requires a lower percentage of a binder, while a more needle shaped crystal requires a higher level of binder to hold the form together.

In one embodiment the coating which is used in the coated particle of the active ingredient is substantially free of a material such as polyethylene glycol which melts below 85° C., in order to prevent damage to the integrity of the coating during the heating step. In this embodiment “substantially free” is defined as less than 2 percent of polyethylene glycol by weight of the dried coating.

In one embodiment, the oral disintegrating tablet further contains one or more effervescent couples. In one embodiment, effervescent couple contains one member from the group consisting of sodium bicarbonate, potassium bicarbonate, calcium carbonate, magnesium carbonate, sodium carbonate and one member selected from the group consisting of citric acid, malic acid, fumaric acid, tartaric acid, phosphoric acid, alginic acid.

In one embodiment, the combined amount of the effervescent couple(s) in the oral dissolving tablet or tablet portion is from about 2 to about 20 percent by weight, such as from about 2 to about 10 percent by weight of the total weight of the orally dissolving tablet portion.

In one embodiment the oral disintegrating tablet or tablet portion is designed to dissolve in the mouth when placed on the tongue in less than about 60 seconds, e.g. less than about 45 seconds, e.g. less than about 30 seconds, e.g. less than about 15 seconds.

In one embodiment the oral disintegrating tablet or tablet portion meets the criteria for Orally Disintegrating Tablets as defined by the draft Food and Drug Administration guidance, as published in April, 2007, incorporated herein by reference. In one embodiment the oral dissolving tablet of this invention meets a two-fold definition for orally disintegrating tablets including the following criteria: 1) that the solid dosage form is one which contains medicinal substances and which disintegrates rapidly, usually within a matter of seconds, when placed upon the tongue and 2) be considered a solid oral preparation that disintegrates rapidly in the oral cavity, with an in vitro disintegration time of approximately 30 seconds or less, when based on the United States Pharmacopeia (USP) disintegration test method for the specific medicinal substance or substances.

Pharmaceutical dosage forms, such as pills, capsules, tablets and the like may be packaged in blister packages, which are comprised of multilayered sheets of material having pockets for containing the dosage forms. Conventional blister packages include packages having a foil layer through which a user of the package must push the tablet, breaking the foil. Hall et al., U.S. Pat. No. 4,158,411, discusses such a blister package. Blisters having open tops for containing pharmaceutical tablets are formed in a flexible sheet of plastic or aluminum material. An optional paperboard layer having disc-shaped punch-outs covers the open tops of the blisters overlying each dosage form. A foil layer covers the paperboard layer, holding the punch-outs in place. To open the package, the user must collapse the blister and push the tablet through the foil, also removing the punch-outs.

Another type of blister package provides perforations between separable blister units so that the user can separate an individual dosage from the package prior to opening. U.S. Pat. No. 4,398,634 to McClosky, illustrates a blister package of this type. The blister portions are defined by tear-resistant, substantially planar plastic sheets sealed to one another in seal zones. The seal zones are located around the periphery of each blister unit, forming pockets of unsealed areas which define the blisters, centrally located in the blister unit. Weakened areas in the seal zones allow the user to separate the blisters into individual units by tearing a unit away from the package. Upon separation of the unit, the user tears through the plastic layers, through the blister, to gain access to the dosage form. A slit in the corner of the unit is provided for easy tearing.

Another type of blister package includes individual units which, upon separation, reveal a tab for opening the blister. U.S. Pat. No. 5,046,618 to Wood discloses this type of blister package. The blister package is formed from a sheet of material having blisters formed therein and a substantially planar lidding sheet. This blister package has two rows of blisters, each blister unit separated from an adjacent unit by perforations. Tear strips separate the rows with perforations that run between the tear strips and the blister units. To open the package, a user separates an individual unit from the package with a tear strip still attached to the unit. This tear strip must be removed to access the tab, which comprises an unsealed area on the corner of the blister unit. After the tear strip is removed, the user grabs the corner of the lidding sheet and peels the sheet back to reveal the dosage form.

There are various production based machines which may be suitable for making blister packaging, including the use of platen sealing such as that made by the Uhlmann Packaging Systems company under model number UPS4 and the use of rotary sealing such as that made by the Bosch Packaging Group company under model number TLT 1400 and the TLT 2800.

The packaged dosage form may be comprised of a blister sheet having a plurality of recesses containing dosage forms arranged, for example, in rows and columns. The type of blister package is not critical to the invention. The blister package includes a plurality of unit packages, each unit package incorporating one recess and a sheet overlying that recess. A set of tear lines can be included between the adjacent unit packages so that a user of the package may tear along the tear lines to separate a unit package.

The recesses of the package and the dosage forms disposed in the recesses may have essentially any shape. For example, the dosage forms may be disk-shaped tablets, oblong capsules, square-shaped pills, hemispheres or truncated cones. Shapes for recesses include circular, oblong, polygonal, triangles or star shapes in the plane of the blister sheet.

Furthermore, the walls and bottom of the recesses may define a shape in the form of a surface of revolution, about a vertical axis normal to the flange surrounding each of the recesses. For example, the recesses may have a curved, cup-like shape. Where the dosage forms are disc-shaped, they may each have an edge which contacts the walls of the recess in which each dosage form is disposed. The edge and walls define an annular region of contact coaxial with the vertical axis of the recess. The edge of such a disc-shaped dosage form may comprise a bevel which contacts the walls of the recess. The annular region of contact prevents shifting of the dosage form within the blister and the damage to the dosage form associated with such shifting. The blister must be substantially deformable to allow for the punch out and removal of the orally disintegrating tablet without breakage of the tablet. The shape of the blister must also be such that the orally disintegrating tablet can be punched out and removed without breakage of the tablet. In one embodiment the (obtuse) angle of the bottom face of the blister to the angle of the side wall of the blister is greater than 90° C., e.g. greater than 110° C.

In one embodiment a lubricant is added to the blister package prior to the addition of the flowable powder. This lubricant may be a liquid or solid, or integrated into the blister material. Suitable lubricants include but are not limited to solid lubricants such as magnesium stearate, starch, calcium stearate, aluminum stearate and stearic acid; or liquid lubricants such as but not limited to simethicone, lecithin, vegetable oil, olive oil, or mineral oil. In certain embodiments the lubricant is added at a percentage by weight of the orally disintegrating tablet of less than 5 percent, e.g. less than 2 percent, e.g. less than 0.5 percent.

A flowable material, preferably in the form of a powder or particulate agglomerate is introduced into each unit of a product holding tray. In one embodiment the powder material can be defined as one with an angle of repose of 20 to 44 degrees. The angle of repose is defined by Terzaghi in “The Theoretical Soil Mechanics in Engineering Practice”, Wiley, N.Y., 1948, as the angle between the horizontal and slope of a heap of soil (or powder) dropped from some elevation. In the embodiments of this invention it is defined as the constant angle to the horizontal assumed by a cone like pile of material. This pile is built from a point above the horizontal using two flat glass plates separated by at least ½ inch and which allows for overflow.

The flowable material is preferably introduced into recesses that are provided in product holding tray that can be a blister-type package described above. The materials in each unit are heated to a temperature and for a period of time to melt the binder partially or substantially throughout the dosage form. The melted binder begins to flow and forms a fused aggregate portion, fusing multiple particles together, and resulting in a unitary dosage form suitable for handling, removal from a blister and ingestion. Other components remain solid and maintain their physical properties, including hardness. The temperature of the recess contents during the heating step should be above the melting point of the binder, but below the melting points and the decomposition temperatures of the other ingredients of the tablet, including the active ingredient. Accordingly, the temperature during heating is typically in the range of about 30 to about 200° C. The time of heating is dependent on the binder and the dimensions of the orally disintegrating form or portion, and must be sufficient in conjunction with the temperature to fuse and stabilize the agglomerate form. In certain cases the active ingredient may be temperature sensitive, requiring different minimal heating temperature with a longer heating time. In one embodiment the temperature may be minimal, e.g. between 40° C. and 70° C. with a heating time greater than 1 hour. In another embodiment the temperature may be higher, e.g. greater than 70° C. with a heating time of less than one hour. In one embodiment the time of heating should be minimal, i.e., on the order of less than about 30 seconds, more preferably less than about 15 seconds.

Suitable heat sources include a radiant heater, conductive heating, convective heating, radiofrequency heating, sonic heating, microwave heating, or laser. The temperature and time of cooling are such as to solidify the melted binder. In one embodiment, the temperature during cooling is about 25° C. to about 0° C., and the time of cooling is about 10 to about 60 seconds. Generally, the higher the temperature during cooling, the longer the cooling time. In one embodiment the cooling takes place at room temperature (25° C.) for greater than 5 minutes.

In one embodiment, an edible form is pre-made prior to the addition of the flowable powder. An outer hard candy or compressed ring is manufactured as an edible form, the fixed amount of flowable powder containing at least one active ingredient is added, and the dosage form is heated for the temperatures and times described above to form an orally disintegrating tablet portion within the dosage form, and subsequently packaged into a blister, pouch or bottle. The edible form must be substantially enclosed in order to hold the powder for the heating or fusing step. In these embodiments substantially enclosed can be achieved by forming a ring, an oval or other shape such as but not limited to a triangle, star, moon, etc. with an internal hollow portion sufficient to hold the powder. This form must be placed onto a surface in order to hold the powder on the bottom portion. This surface may be suitable for holding any flat shape including but not limited to plastic, metal, or composite. This may also be achieved within a preformed blister package and may have negative embossing in order to transfer a logo, image or product identification upon heating and fusing of the dosage form. Alternatively, the dosage form may be lasered or printed for aesthetic imaging (shapes, characters, colors, etc.) or identification (product name, dosage, etc.).

The outer hard candy form may be made using uniplast rolling, roping and subsequent cutting and stamping, as well as depositing into molds. The hard candy portion contains one or more sugars selected from the group consisting of isomalt, sucrose, dextrose, corn syrup, lactitol, and lycasin. In one embodiment, the hard candy portion contains at least 50% (such as at least 75%, such as at least 90%) by weight of such sugar(s).

In one embodiment the outer edible form contains one portion of at least one active ingredient and the orally disintegrating tablet inner portion contains a second portion of the same active ingredient that is in the outer edible form. In one embodiment the outer edible form contains one portion of at least one active ingredient and the orally disintegrating tablet inner portion contains a second portion of a different active ingredient than is in the outer edible form. In one embodiment the outer edible form disintegrates at a rate of at least 10 times the rate of the orally disintegrating tablet inner portion. The first and second portions can be the same or different.

In one embodiment, the dosage form comprising an outer edible form and an inner orally disintegrating tablet inner portion is coated with an immediate release sugar coating or film coating. To produce such a dosage form, the step following the fusing (heating) and subsequent cooling of the dosage form would involve further sugar or film coating in a coating pan.

Specific embodiments of the present invention are illustrated by way of the following examples. This invention is not confined to the specific limitations set forth in these examples, but rather to the scope of the appended claims. Unless otherwise stated, the percentages and ratios given below are by weight.

### EXAMPLE 1

### Cold Forming of Blister

a) Using a Bosch TLT 1400 (rotary thermoforming sealing) blister line machine, a web of aluminum blister forming material is unwound from a roll and heated to a pre-determined temperature. The heated material is then indexed into the forming station where compressed air and/or a vacuum is used to form cavities in the web at a ⅝ inch flat round cavity with depressions containing a “TY” as an identifier to produce a thermoformed web.
b) The resulting thermoformed web is indexed into a feeder station where formulations described below are deposited into the formed cavities.

### EXAMPLE 2

### Orally Disintegrating Immediate Release Loratidine Tablet Blend

TABLE 1
Tablet Blend Formulation:

| Granulation | Trade Name | Manufacturer | G/Batch | Mg/Tablet |
|---|---|---|---|---|
| Maltodextrin | Maltrin QD M600 | | 9.9 | 832.7 |
| Polyethylene | Polyglycol | | 5.0 | 421.3 |
| Glycol | 4000 PF | | | |
| Erythritol | Eridex 16951 | | 2.5 | 210.7 |
| | powder | | | |
| Sucralose USP | Sucralose | | 0.1 | 8.4 |
| Flavor | | | 0.2 | 16.9 |
| Loratidine | | | 0.007 | 10.0 |
| Total | | | 17.8 | 1500.0 |

Manufacturing Process:

Maltodextrin, erythritol, sucralose and flavor are screened through a 30 mesh screen and placed into a 100 cc plastic bottle and mixed end-over-end for 5 minutes. The blend is then filled into the pre-formed blister cavities in Example 1, and placed into a convection oven set at 85° C. for 15 minutes. Blister forming pins or punches used to pre-form the blister cavities prior to addition of the powder blend contain small injection ports which inject approximately about 0.1-5 mg of soy lecithin onto the surface of the blister upon forming the cavity, in order to facilitate ejection of the final dosage form. The formed blister material from Example 1 is then indexed into a seal station where a foil lidding is applied. The lidding material is unwound from a roll and sealed together using heat and mechanical pressure resulting in the product being contained within the cavity.

The sealed web is indexed toward the perforating station. The perforating station uses sharp cutting blades to place perforations through the web resulting in a blister card with an opening feature. Lastly, the web moves to the punch station where individual blister are cut from the web into individual cards containing 6 orally disintegrating forms per card.

The blister cavity is then cooled at 0° C. for 5 minutes and sealed. The tablets are then removed from the blister cavity as a single dosage unit for ingestion.

### EXAMPLE 3

### Preparation of an Edible Outer Ring Portion

All materials set forth in Table 2 below are manually passed through a 30 mesh screen. One and a half (1.5) kg of the resulting blend are placed in a 4 quart V-Blender and mixed for 5 minutes.

TABLE 2
Components of Compressed Edible Outer Ring Portion Blend

| | Percent | |
|---|---|---|
| Ingredients | (w/w) | mg/hard candy portion |
| Sorbitol | 5.00 | 50.0 |
| Compressible Sucrose* | 92.75 | 927.5 |
| Menthol | 1.00 | 10.0 |
| Peppermint Flavor | 0.50 | 5.0 |
| Magnesium Stearate | 0.75 | 7.5 |
| TOTAL | 100.0 | 1000.0 |

*Commercially available from Domino Specialty Ingredients, Baltimore, MD

Four hundred (400) g of the resulting blend is then removed from the blender and compressed on a rotary tablet press at 60 rpm using ¾″ ringed tablet tooling in order to yield flat faced rings having ½″ empty centers and having a weight of 1000 mg and a hardness range of not less than 15 kp/cm², and a thickness of about 0.20 inches.

### EXAMPLE 4

### Preparation of Outer Edible Ring with Fused Orally Disintegrating Tablet Inner Portion

TABLE 4
Fused Orally Disintegrating Tablet Inner Portion Blend Formulation:

| Granulation | Trade Name | Manufacturer | Percent | Mg/Tablet |
|---|---|---|---|---|
| Maltodextrin | Maltrin QD | | 54.8 | 277.6 |
| | M600 | | | |
| Polyethylene | Polyglycol 4000 | | 27.7 | 140.4 |
| Glycol | PF | | | |
| Erythritol | Eridex 16951 | | 13.9 | 70.2 |
| | powder | | | |
| Sucralose USP | Sucralose | | 0.6 | 2.8 |
| Flavor | | | 1.1 | 5.6 |
| Loratadine | | | 2.0 | 10.0 |
| Total | | | 100 | 506.6 |

Manufacturing Process:

Maltodextrin, erythitol sucralose and favor are screened through a 30 mesh screen and placed in a 100 cc plastic bottle and mixed end-over-end for 5 minutes. The blend is then filled into the outer edible ring portions from Example 3 sitting on a flat PVC plastic sheet, and placed into a Convection Oven set at 85° C. for 15 minutes. The blister cavity is then cooled at 0° C. for 5 minutes and sealed. The dosage forms are then removed from the blister cavity as a single dosage unit for ingestion.

I/we claim:

1. A process for making an orally disintegrating dosage form comprising a granular matrix comprising:
a) providing a unit product sheet having a recess in a desired shape and volume suitable for containing said dosage form;
b) introducing a predetermined amount of a flowable powder blend matrix containing a binder having a melting point of about 20-160° C. in the recess;
c) heating the contents in the recess to a temperature above the melting point of the binder and for a sufficient period of time to melt and cause the binder to fuse into an aggregate within and throughout the dosage form, and
d) cooling the fused dosage form in the recess so that the dosage form solidifies into the orally dissolving dosage form suitable for consumption.

2. The process of claim 1 wherein said dosage form has a hardness of less than 1000 grams as measure using Texture Analyser TA-XT2i that is fitted with a 7 millimeter diameter flat faced probe.

3. The process of claim 1 wherein said dosage form disintegrates in less than 30 seconds.

4. The process of claim 1 wherein the unit dosage package is a blister-type package that comprises aluminum.

5. The process of claim 1 wherein the heat is applied via convection, conduction, sonic heating, radiofrequency, laser, infrared, microwave.

6. The process of claim 1 wherein said binder is selected from the group consisting of fats, waxes, water soluble polymers, long chain alcohols and their derivatives, and mixtures thereof.

7. The process of claim 1 wherein at least 95% by weight of the water soluble binder has a particle size of less than 100 microns.

8. The process of claim 1 wherein the recess has positive imprinted portions on its interior surface and which produce corresponding patterns in the final dosage form.

9. The process of claim 8 wherein the positive imprinted portions are in the form of a design, logo or marking.

10. An orally disintegrating tablet made by the process of claim 1.

11. An orally disintegrating tablet of claim 10 wherein the tablet binder comprises less than 40% of the dosage form.

12. A process for making a dosage form comprising an edible outer portion and an inner orally disintegrating dosage form comprising a granular matrix comprising:
a) preparing an edible outer form having a recess in a desired shape and volume suitable for containing the orally disintegrating portion of said dosage form;
b) introducing a predetermined amount of a flowable powder blend—containing matrix containing a binder having a melting point of about 20-160° C. in the recess;
c) heating the contents in the recess to a temperature above the melting point of the binder and for a sufficient period of time to melt and cause the binder to fuse into a continuous phase within and throughout the dosage form, and
d) cooling the fused dosage form in the recess so that the dosage form solidifies into the orally dissolving dosage form suitable for consumption.

13. The process of claim 12 wherein the edible outer form is prepared via compression.

14. The process of claim 12 wherein the edible outer form is a outer hard candy form prepared by a method selected from the group consisting of uniplast rolling, roping and subsequent cutting and stamping, or mold depositing.
